(19) United States
(12) Patent Application Publication
(10) Pub. No.: US 2001/0039020 A1
(43) Pub. Date: Nov. 8, 2001

(54) METHOD AND DEVICE FOR NONSYNTHETIC DECONVOLUTION

(76) Inventors: RONALD ZUCKERMANN, BERKELEY, CA (US); MANOJ DESAI, PLEASANT HILL, CA (US); GAVIN DOLLINGER, SAN FRANCISCO, CA (US); TIMOTHY DAWES, CLAYTON, CA (US); PETAR STOJADINOVIC, EMERYVILLE, CA (US)

Correspondence Address: Chiron Corporation, Intellectual Property - R440, P.O. Box 8097, Emeryville, CA, 94662-8097, US

(21) Appl. No.: 09/306,700

(22) Filed: May 6, 1999

Related U.S. Application Data
(60) Provisional application No. 60/084,843, filed on May 8, 1998.

(51) Int. Cl. G01N 33/53; G01N 33/567

(52) U.S. Cl. 435/7.1; 435/7.2; 436/501; 436/518; 422/63; 422/81; 422/100; 422/102

(57) ABSTRACT

A method is provided for use in solid phase chemical synthesis such as in the synthesis of polypeptides, peptoids, and other molecules synthesized by solid phase methods. The method is used to identify compounds having activity against a selected target, wherein the compounds are present in a mixture obtained from a combinatorial library. A bead distributor probe is also provided. The probe is used to extract beads from a population of beads, and then deliver the bead to a selected location. A capillary bead insert is also provided, as well as a bead distribution system which includes both a bead distributor probe and a capillary bead insert.

### CROSS-REFERENCE TO RELATED APPLICATION

This application is related to provisional patent application Ser. No. 60/084,843, filed May 8, 1998, from which priority is claimed under 35 USC §119(e)(1) and which is incorporated herein by reference in its entirety.

### TECHNICAL FIELD

The invention relates generally to solid phase chemical synthesis. More particularly, the invention relates to the handling of bead-based combinatorial libraries, and to a novel method and apparatus for use in deconvolution of libraries of polypeptides, peptoids, cyclic or heterocyclic organic compounds, and other solid phase organic molecules.

### BACKGROUND OF THE INVENTION

Individual polymers or oligomers of amino acids or the like can be readily prepared using conventional solid phase synthetic technologies. For example, a single defined polypeptide can be synthesized using Merrifield solid phase synthetic schemes. Merrrifield, J. Am. Chem. Soc. 85:2149-2154 (1963); Tam et al., The Peptides, Academic Press (New York), pp. 185-249 (1987). Another well-known method for achieving solid-phase peptide synthesis uses 9-fluorenylmethoxycarbonyl (Fmoc) protecting groups on the amino acids (Meienhofer et al., Int. J. Pept. Protein Res. 13:35 (1979), Atherton et al., Bioorg. Chem. 8:351 (1979)). In this technique, the peptide is immobilized on any of a wide variety of commercially available polystyrene resins (Wang, S., J. Am. Chem. Soc. 95:1328 (1973), Mergler et al., Tetrahedron Lett. 29:4005 (1988), Albericio et al., Int. J. Pept. Protein Res. 30:206 (1987)).

Methods for the systematic synthesis of a multiplicity of polymers to screen for pharmacological or biological activity have also been developed. Particularly, combinatorial libraries can be prepared containing a large number of polymers using “resin-splitting” or “mix/split” techniques. Furka et al., Int. J. Peptide Protein Res. 37:487-493 (1991); Lam et al., Nature 354:82-84 (1991). Resin-splitting strategies have also been used to generate mixtures of lower complexity to study ligand-receptor binding activity and enzyme activity structure-activity relationships. Zuckermann et al., Proc. Natl. Acad. Sci. USA 89:4505-4509 (1992); Peuthory et al., Proc. Natl. Acad. Sci. USA 88:11510-11514 (1991). Methods for producing libraries of cyclic or heterocyclic organic compounds using resin-splitting procedures have also been described, for example, in International Publication No. WO 96/40201 which enjoys common ownership herewith.

Synthesis of such combinatorial libraries allows for the generation of many diverse molecules in parallel, e.g., bulk populations containing from 2 or several components up to 10⁶ or more components, which molecules can then be screened against pharmacologically relevant targets. Generally, synthesis is carried out using resin supports (beads) where each bead supports a single unique compound and is present in a mixture of beads supporting other related compounds. The molecules can be synthesized with or without identifier tags to assist in deconvolution. Once a library mixture has been identified as having a desired activity, steps can be taken to identify the specific active component(s) from the library, and the chemical structure is ascertained using iterative deconvolution techniques (e.g., resynthesis).

### SUMMARY OF THE INVENTION

It is a primary object of the invention to provide a method for identifying one or more active compounds from a combinatorial library, wherein the identified compounds have activity against a selected target and the identification is carried out without having to resort to resynthesizing the compounds. The method comprises the following steps (a) providing a mixture of compounds from a combinatorial library. The mixture is generally comprised of a plurality of resin support beads having compounds attached thereto, wherein each bead has only one discrete compound attached thereto; (b) individually distributing beads from the mixture provided in step (a) into a plurality of reaction vessels such that each vessel contains a single bead; (c) cleaving the compounds from the beads and separating the beads away from the cleaved compounds, thereby providing discrete samples of individual compounds; (d) screening a portion of each cleaved compound for activity against a selected target to identify active compounds; and (e) performing chemical analyses on a reserved portion of the active compounds to chemically identify active compounds from the mixture.

In one aspect of the invention, a bead distributor probe is used to individually distribute the beads in the above method. The bead distributor probe uses vacuum to select discrete beads from the mixture of beads and then uses a gas discharge to deliver the selected beads to a selected location, for example, into an array of reaction vessels. In other aspects, the mixture is a sublibrary of a combinatorial library, wherein the sublibrary contains about 20-100 discrete compounds.

In one particular embodiment of the invention, the mixture of beads provided in step (a) of the above method is divided into an archive portion and a screening portion, and a preliminary screening step is used to assess the screening portion to determine if the mixture contains one or more active compounds prior to performing steps (b)-(e) on a sample obtained from the archive portion. If desired, the resin support beads present in the archive portion can be maintained in dried form. Furthermore, the archive portion can be distributed into a plurality of replica arrays, wherein each array contains a sufficient number of beads to provide a greater than 95% probability that every compound in the mixture is represented in the array.

It is also an object of the invention to provide a bead distributor probe. The probe comprises the operative combination of (a) an elongate tube having an upper end, a lower end, and a lumen extending therethrough; (b) means for communicating the upper end of the tube with an associated source of vacuum and an associated gas delivery means; and (c) means for switchably communicating the tube with (i) the source of vacuum to establish a vacuum in the lumen of the tube, and (ii) the gas delivery means to deliver gas through the lumen of the tube, wherein the lower end of the tube is adapted for extracting a single bead from a slurry of beads when vacuum is established in the lumen and for delivering the bead to a selected location when gas is delivered through the lumen.

In one embodiment, the above-described bead distributor probe is configured for use with conventional combinatorial chemistry solid bead supports. In particular, a probe is provided wherein the lower end of the tube is adapted for extracting a single bead from a slurry of beads. The beads preferably have a substantially uniform diameter which can range from about 50 μm to 2 mm. The use of a substantially uniform population of beads in the invention provides the added benefit that final reaction volumes of compounds cleaved from the beads will have substantially uniform compound concentrations.

It is a still further object of the invention to provide a capillary bead insert. The capillary bead insert comprises (a) an elongate outer sleeve having a closed bottom end and a solvent reservoir arranged at an open top end thereof, wherein the solvent reservoir has a larger inner diameter than the inner diameter of the bottom end of the outer sleeve; and (b) an elongate inner sleeve which is adapted for placement within the outer sleeve. The inner sleeve has a bottom portion, an intermediate portion, and a top portion. The bottom portion of the inner sleeve has an outer diameter that is slightly less than the inner diameter of the bottom end of the outer sleeve, and an opening in said bottom portion provides fluid communication between the inner and outer sleeves when the inner sleeve is placed within the outer sleeve. The intermediate portion of the inner sleeve has a substantially reduced inner diameter relative to the inner diameter of the bottom portion of the inner sleeve, and an open bead cup is arranged at the top portion of the inner sleeve.

It is still a further object of the invention to provide a bead distribution system which comprises the capillary bead insert and the bead distributor probe of the present invention.

Additional objects, advantages and novel features of the invention will be set forth in part in the description that follows, and in part will become apparent to those skilled in the art upon examination of the following, or may be learned by practice of the invention.

### BRIEF DESCRIPTION OF THE FIGURES

FIGS. 1A-1C depict a bead distributor probe, and provide a pictorial representation of the use thereof in an automated system for extracting individual beads from a combinatorial library, and dispensing the same into a suitable container.

FIG. 2 depicts a capillary bead insert constructed according to the present invention.

### DETAILED DISCLOSURE OF THE INVENTION

The practice of the methods of the present invention will employ, unless otherwise indicated, conventional techniques of synthetic organic chemistry, including solid-phase synthesis, peptide synthesis, polysaccharide synthesis, and other solid phase organic chemistries, that are within the skill of the art. Such techniques are explained fully in the literature. See, e.g., Thompson et al., “Synthesis and Applications of Small Molecule Libraries,” Chem Rev. 96:55-600 (1996); Terrett et al., “Combinatorial Synthesis—The Design of Compound Libraries and Their Application to Drug Discovery,” Tetrahedron 51(30):8135-8173 (1995); Kirk-Othmer's Encyclopedia of Chemical Technology; House's Modern Synthetic Reactions; C. S. Marvel and G. S. Hiers' text, ORGANIC SYNTHESIS, Collective Volume 1; Oligonucleotide Synthesis (M. J. Gait, ed., 1984); and Bunin, B., “Combinatorial Index,” Acad. Press (1998).

All patents, patent applications, publications and other types of references cited herein, whether supra or infra, are hereby incorporated by reference in their entirety.

Definitions:

Before the present invention is disclosed and described in detail, it is to be understood that this invention is not limited to specific assay formats, materials or reagents, as such may, of course, vary. It is also to be understood that the terminology used herein is for the purpose of describing particular embodiments only and is not intended to be limiting.

It must be noted that, as used in the specification and the appended claims, the singular forms “a,” “an” and “the” include plural referents unless the context clearly dictates otherwise. Thus, for example, reference to “a reaction vessel” includes two or more such vessels, and the like.

In this specification and in the claims which follow, reference will be made to a number of terms which shall be defined to have the following meanings:

The terms “solid phase,” “resin support bead,” and “bead,” intend any solid support or substrate on which the reaction steps of chemical syntheses involving a sequence of reaction steps can be carried out. Thus, the term includes particulate substrates such as polystyrene resins which have traditionally been employed in standard Fmoc chemical syntheses.

The term “library” or “combinatorial library” includes, inter alia, a collection of sublibraries each containing 2-500 components or compounds, and more preferably about 10-100 components or compounds. The components or compounds of such sublibraries are diverse synthesized molecules which have been prepared using standard combinatorial chemistries (see, e.g., Furka et al., Int. J. Peptide Protein Res. 37:487-493 (1991); and Lam et al., Nature 354:82-84 (1991)).

In one embodiment of the invention, a method is provided for screening components of a combinatorial library for relevant biological and/or pharmacological activity, and then performing a nonsynthetic deconvolution to identify and characterize specific components from the library. A combinatorial library is constructed (e.g., using a conventional mix/split synthesis on suitable resin bead supports) which comprises a number of sublibrary mixtures, each generally containing about 2 to 500, and preferably about 20 to 100 compounds each. It is preferable that the bead supports be high-loading beads (which provide >1 nmole of compound per bead). It is also preferable that the bead supports have a substantially uniform diameter. The use of a substantially uniform population of bead supports in the methods of the invention provide the added benefit that final reaction volumes of compounds cleaved from the bead supports will have substantially uniform compound concentrations. Thus, the bead supports preferably have a diameter variance of about 40%, preferably about 30%, more preferably about 20%, and most preferably about 510% or less. The actual number of individual compounds in each sublibrary is not important or limiting in the present invention, and the method can be practiced with any size sublibrary selected according to user preferences. Prior to cleavage of the compounds from the resin bead supports, each sublibrary is split into archive and screening samples, wherein the screening sample is generally comprised of roughly 10 to 30 percent of the entire sublibrary volume.

A small aliquot of the archive sample can be used in a statistical post-synthesis analysis, wherein the method and device of the present invention are used to deposit single beads in a suitable reaction vessel (preferably a multi-well plate or a fixed array of reaction vials) so that each bead can be chemically analyzed or screened separately. This statistical analysis can be used to determine the amount of, and/or identify different compounds present in the archive sample. The remainder of the archive sample is retained in bound form (uncleaved), but is treated to remove solvents, suitably dried, and then stored either as an intact archive sample, or in a plurality of replica samples which can contain individual beads, small collections of beads, or the entire sublibrary pool of beads. As will be understood by the ordinarily skilled artisan upon reading the instant disclosure, storing the archive sample in a dried, uncleaved form allows for indefinite archiving of the library with a reduced incidence of compound loss and/or decomposition.

The screening sample is distributed into reaction vessels (e.g., a multiwell plate or an array of reaction vials) to establish screening aliquots. The screening aliquots are then treated in a suitable cleavage step to remove and separate the bead supports from the cleaved compounds, and the cleaved compounds are screened in a typical primary screen for desired activity. For example, the cleaved compounds can be subjected to evaporation to remove solvents, lyophilized, labelled (if desired), and subjected to dissolution. Sublibraries which contain active components are then subjected to the following nonsynthetic deconvolution methodology.

The dried archive sample, which corresponds to a sublibrary identified as having activity in the above-described primary screen of the analysis sample, is then retrieved. The sample is reconstituted in a suitable solvent, preferably a solvent with a density of at least about 1.1 g/ml, and a suitable bead-sorting apparatus is used to distribute one bead per well in a multiwell reaction plate or reaction vessel array in multiple redundancy such that there is a greater than 95% probability that every compound in the sublibrary is represented (e.g., at a 5× redundancy). If desired, the bead-sorting apparatus is used to distribute any number of beads per well, such as where combinations of compounds are to be assessed for activity in the screening method.

After the desired number of beads have been distributed, the nonsynthetic deconvolution method of the invention is then carried out. As discussed above, each sublibrary generally contains about 20-100 compounds each, thus about 100-500 discrete beads can be distributed from the archive sample to provide a screening array with adequate compound representation. The compounds are cleaved from the bead supports using a suitable cleavage reagent, and the compounds reconstituted in a suitable reaction solvent (e.g., DMSO). Portions of the cleaved compounds are delivered into a further array which replicates the screening array. This replica array is then contacted with the selected target, and biologically or chemically active compounds are identified using conventional screening techniques readily available to the ordinarily skilled artisan. A sampling of the reserved portion of the screening array (e.g., about 10%) is then removed for conventional chemical analytics (e.g., liquid chromatography such as HPLC, mass spectrometry (MS) and/or nitrogen (N₂) analyses) in order to provide for direct chemical identification and characterization of active compounds. As can be seen, the above nonsynthetic deconvolution obviates the iterative deconvolution by resynthesis normally needed to identify single compounds responsible for biological and/or chemical activity in a mixture of compounds that were synthesized by a mix-and-split method. If desired, the individual compounds can be suitably labeled with a chemical tag (e.g., mass tags, enzymatic labels, or the like) to facilitate sample identification, however such labeling only provides marginal advantage in the present nonsynthetic deconvolution method, since MS data can easily be used as a “tag” to identify active sublibrary components.

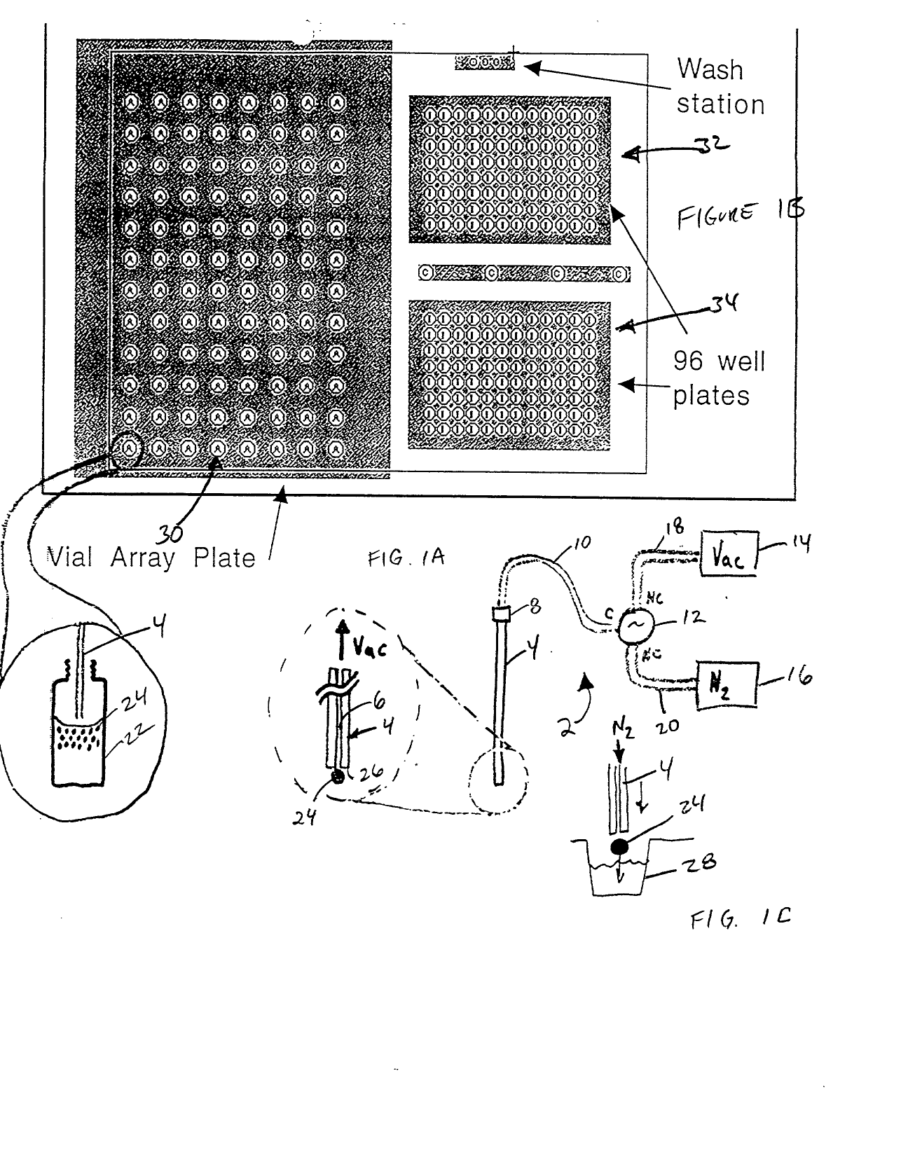

In another embodiment of the invention, a bead distributor probe is provided which allows for the accurate selection of individual resin support beads from a bead suspension and the placement thereof into a suitable reaction vessel. Referring to FIGS. 1A-1C, and particularly to FIG. 1A, the bead distributor probe is generally indicated at 2. The probe includes an elongate tube 4 having a lumen 6 extending therethrough. The actual diameter of the lumen can vary widely, and is selected for use with beads of a particular size, wherein the lumen diameter is generally about 20-40% of the bead diameter. The beads which are used in the practice of the above-described methods generally range from about 50 μm to 2 mm in diameter, and preferably about 150-500 μm in diameter. The tube 4 can be comprised of any suitable material that is sufficiently resistant to common organic solvents. For example, the tube can be formed from a glass (fused silica) or stainless steel capillary tube of suitable bore, strength, and overall size. Furthermore, the tube 4 can include a head disposed on the tip 26 thereof, wherein the head is comprised of a material which resists electrostatic or hydrostatic attraction between the tube 4 and resin beads. For example, the head can be comprised of a suitably inert polymer such as poly(tetrafluoroethylene) (commercially available, for example, under the tradename TEFLON@).

An upper end 8 of the tube 4 is connected to a conduit 10 that provides for communication with a multi-position valve 12, which in turn is operably connected to a suitable control means, a source of vacuum 14, and a gas delivery means 16 via conduits 18 and 20, respectively. The gas provided by the gas delivery means is preferably an inert gas, for example nitrogen. If desired, the valve 12 can also be connected to a syringe pump which allows for dispensing of reagent or washing liquids from the tube 4. These liquids can also be used to agitate or mix the contents of the reaction vessel or wash station.

Referring to FIGS. 1B-1C, the bead distributor probe 2 is used as follows. After combinatorial chemistries have been carried out to provide one or more synthesized libraries 30 of molecules, the probe 2 is used to select individual beads 24 (each of which support individual compounds) from a reaction vessel 22 containing a suspension of beads (bead slurry). The slurry comprises the beads in a dense solvent (e.g., dichloroethane or chlorobenzene) so that the beads form a layer at or near the meniscus. That is, the valve 12 is switched to a first position to provide communication between the vacuum source 14 and the tube 4. The tip 26 of the tube is then lowered into the reaction vessel 22 and contacted with the bead slurry to select a single bead 24. Typically, the tip 26 is lowered about 0.5 to 1.0 mm below the meniscus. The vacuum is sufficient to allow the tip 26 of the tube to grip and retain the bead, and the bead can then be extracted from the reaction vessel. The tube is then moved into position over a suitable container 28, such as a well in a multiwell plate or a member of an array of suitable vessels. The container 28 typically contains a solvent to prevent the bead from sticking to the probe tip, and the tip is lowered into the solvent. The valve 12 is then switched to a second position to provide communication between the gas delivery means 16 and the tube 4, and a low pressure gas discharge from the gas delivery means is used to deposit the bead 24 into the container 28.

The bead distributor probe 2 can be operated manually, or operated by an automatic control arm in order to sample beads from combinatorial libraries. If an automated system is employed, the tube 4 can be held by a robotic arm which positions the tube over one or more library reaction vessels, and then moves the tube between the reaction vessel and an analysis array. Positioning of the robotic arm is controlled by any suitable microprocessor control means, which is also used to move the switch 12 between vacuum (bead extraction), gas discharge (bead delivery), and, if desired, liquid discharge positions.

Referring to FIG. 1B, if an automated system is used to control bead sampling, one or more sublibraries will be arranged in an array 30 at an addressable location (e.g., X-Y coordinate), and the robotic arm will move the tube between the combinatorial library array 30 and an analysis array 34 which contains a plurality of addressable analysis locations (e.g., wells in a multiwell plate). The automated system can also move the probe to an optional wash station 32 after bead delivery in order to clean the probe of any residual beads stuck to the probe, and to expel any bead fragments that may have lodged in the lumen of the probe tip.

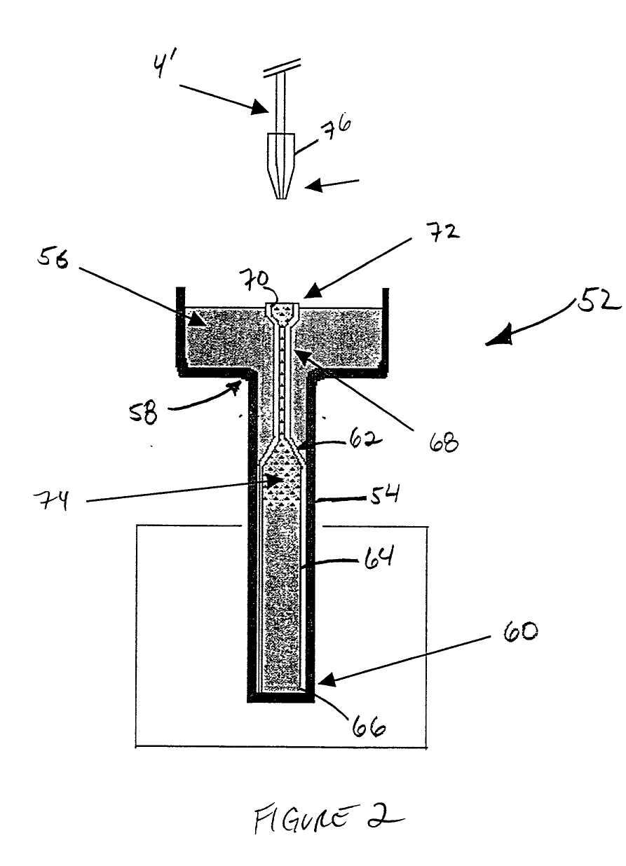

In yet another embodiment of the invention, a capillary bead insert is provided. The insert is configured for use with the bead distributor probe of the present invention. Referring now to FIG. 2, a capillary bead insert is generally indicated at 52. The insert has an elongate outer sleeve 54 which is closed at a bottom end 60 thereof to establish a container. The outer sleeve 54 also has a solvent reservoir 56 arranged at the top end 58 thereof, wherein the solvent reservoir has a larger inner diameter than the inner diameter of the bottom end 60.

The capillary bead insert 52 also comprises an inner sleeve 62 which is adapted to be placed within the outer sleeve 54 of the bead insert 52. More particularly, the inner sleeve 62 has an outer diameter sized to fit through the top end 58 of the outer sleeve, and a length sized to extend substantially to the bottom end 60 of the outer sleeve. The inner sleeve has a bottom portion 64 which has an outer diameter which is just slightly less than the inner diameter of the bottom end 60 of outer sleeve 54. An opening 66 at the lower terminus of the bottom portion 64 of the inner sleeve provides fluid communication (e.g., passage of beads and liquids) between the inner and outer sleeves. The inner sleeve 62 also has an intermediate portion 68 which has a substantially reduced inner diameter relative to the inner diameter of the bottom portion 64 of inner sleeve 62. As will be described below, the inner diameter of the intermediate portion 68 is sized to be about 10-20% larger than the overall diameter of the largest resin bead support used in a particular combinatorial synthesis. The inner sleeve 62 further includes a bead display cup 70 arranged at the top portion 72 thereof.

In use, a suspension of beads 74 (e.g., a slurry formed from an analysis sample mixture of resin beads and a suitable solvent) is placed into the outer sleeve so that it is approximately half full of slurry. The inner sleeve 62 is then lowered into the outer sleeve 54 such that all of the beads become trapped within the inner sleeve 62. The solvent level is increased to a level just below the top of the bead display cup 70 by adding solvent to reservoir 56. The beads then float up through the inner sleeve, wherein the restriction provided by the reduced inner diameter of the intermediate portion 68 causes the beads 74 to travel up the inner sleeve in single file. The bead display cup is sized to accommodate the tip 76 of the tube 4′ of a bead distributor probe (as described above). Individual beads can then be extracted from the slurry, and distributed as also described hereinabove.

It is to be understood that while the invention has been described in conjunction with the preferred specific embodiments thereof, that the foregoing description is intended to illustrate and not limit the scope of the invention. Other aspects, advantages and modifications within the scope of the invention will be apparent to those skilled in the art to which the invention pertains.

We claim:

1. A method for identifying compounds having activity against a selected target, said method comprising:
(a) providing a mixture of compounds from a combinatorial library, wherein (i) said mixture comprises a plurality of resin support beads having compounds attached thereto, and (ii) each said bead has only one discrete compound attached thereto;
(b) individually distributing beads from the mixture provided in step (a) into a plurality of reaction vessels such that each vessel contains a single bead;
(c) cleaving the compounds from the beads and separating said beads from the cleaved compounds, thereby providing discrete samples of individual compounds;
(d) screening a portion of each cleaved compound for activity against a selected target to identify active compounds; and
(e) performing chemical analyses on a reserved portion of said active compounds to chemically identify said compounds.

2. The method of claim 1, wherein the beads are individually distributed using a bead distributor probe which uses vacuum to select discrete beads from a mixture of beads and then uses a gas discharge to deliver the selected beads into a reaction vessel.

3. The method of claim 1, wherein step (b) entails distributing discrete beads into an array of reaction vessels.

4. The method of claim 1, wherein the mixture is a sublibrary of the combinatorial library, and said sublibrary contains about 20-100 discrete compounds.

5. The method of claim 1, wherein the mixture of beads provided in step (a) is divided into an archive portion and a screening portion, and a preliminary screening step is used to assess said screening portion to determine if the mixture contains one or more active compounds prior to performing steps (b)-(e) on a sample obtained from said archive portion.

6. The method of claim 5, wherein the preliminary screening step comprise cleaving the compounds from the beads and contacting the cleaved compounds with a selected target to determine if the mixture contains one or more compounds which are active against said target.

7. The method of claim 5, wherein the resin support beads present in the archive portion are maintained in dried form.

8. The method of claim 7, wherein the archive portion is distributed into a plurality of replica arrays, each said array containing a sufficient number of beads to provide a greater than 95% probability that every compound in the mixture is represented in the array.

9. A bead distributor probe, comprising the operative combination of:
(a) an elongate tube having an upper end, a lower end, and a lumen extending therethrough;
(b) means for communicating the upper end of the tube with an associated source of vacuum and an associated gas delivery means; and
(c) means for switchably communicating the tube with (i) the source of vacuum to establish a vacuum in the lumen of the tube, and (ii) the gas delivery mean to deliver gas through the lumen of the tube, wherein the lower end of the tube is adapted for extracting a single bead from a slurry of beads when vacuum is established in the lumen and for delivering the bead to a selected location when gas is delivered through the lumen.

10. The bead distributor probe of claim 9, wherein the lower end of the tube is adapted for extracting a single bead from a slurry of beads having an average diameter ranging from about 50 μm to 2 mm.

11. A capillary bead insert, comprising:
(a) an elongate outer sleeve having a closed bottom end and a solvent reservoir arranged at an open top end thereof, wherein said solvent reservoir has a larger inner diameter than the inner diameter of said bottom end of said outer sleeve; and
(b) an elongate inner sleeve adapted for placement within the outer sleeve, wherein said inner sleeve has a bottom portion, an intermediate portion, and a top portion, wherein (i) said bottom portion of the inner sleeve has an outer diameter that is slightly less than the inner diameter of the bottom end of the outer sleeve, and an opening in said bottom portion provides fluid communication between the inner and outer sleeves when the inner sleeve is placed within the outer sleeve, (ii) said intermediate portion of the inner sleeve has a substantially reduced inner diameter relative to the inner diameter of the bottom portion of the inner sleeve, and (iii) an open bead cup is arranged at the top portion of the inner sleeve.

12. A bead distribution system, comprising the capillary bead insert of claim 10 and a bead distributor probe configured for use with said capillary bead insert, wherein said probe comprises the operative combination of:
(a) an elongate tube having an upper end, a lower end, and a lumen extending therethrough;
(b) means for communicating the upper end of the tube with an associated source of vacuum and an associated gas delivery means; and
(c) means for switchably communicating the tube with (i) the source of vacuum to establish a vacuum in the lumen of the tube, and (ii) the gas delivery means to deliver gas through the lumen of the tube, wherein the lower end of the tube is adapted for extracting a single bead from a slurry of beads present in the open bead cup of said capillary bead insert when vacuum is established in the lumen, and for delivering the bead to a selected location when gas is delivered through the lumen.
